(19) United States
(12) Patent Application Publication
(10) Pub. No.: US 2023/0121113 A1
(43) Pub. Date: Apr. 20, 2023

(54) PORTABLE RESPIRATOR

(71) Applicant: ITT Manufacturing Enterprises LLC, Wilmington, DE (US)

(72) Inventors: A. David SANDIFORD, Valencia, CA (US); Steve E. KIM, Valencia, CA (US); Mostafa DONYANAVARD, Santa Clarita, CA (US); Matthew T. BLOMQUIST, Lancaster, CA (US)

(73) Assignee: ITT Manufacturing Enterprises LLC, Wilmington, DE (US)

(21) Appl. No.: 17/909,447

(22) PCT Filed: Mar. 25, 2021
(86) PCT No.: PCT/US2021/024034

§ 371 Date: Sep. 6, 2022

Related U.S. Application Data
(60) Provisional application No. 63/004,360, filed on Apr. 2, 2020.

(51) Int. Cl. A61M 16/06; A61M 16/00; A61M 16/04; A61M 16/12; A61M 16/08

(52) U.S. Cl. CPC A61M 16/06; A61M 16/024; A61M 16/04; A61M 16/125; A61M 16/0816; A61M 16/0875; A61M 16/0084; A61M 2205/0216; A61M 2202/0208; A61M 2205/103; A61M 2205/3553; A61M 2205/50; A61M 2205/8206; A61M 2202/0007

(57) ABSTRACT

A portable respirator as described may include an inflatable bag and a respirator body. The inflatable bag may be arranged to provide air to a patient through a face mask or an endotracheal tube in response to compression/retraction actions applied to the inflatable bag. The respirator body may include a first cover portion and a second cover portion arranged to enclose moving components of the portable respirator and define a substantially circular opening for the inflatable bag to be fitted through, a motor, a pair of levers positioned on opposing sides of the substantially circular opening, and a cam mechanically coupled to the motor and arranged to move the pair of levers in response to a rotation action by the motor such that the pair of levers apply the compression/retraction actions to the inflatable bag fitted through the opening.

### BACKGROUND

This application claims priority to U.S. patent application Ser. No. 63/004,360 filed on Apr. 2, 2020. The disclosures of the provisional application are hereby incorporated by reference for all purposes.

### BACKGROUND

Unless otherwise indicated herein, the materials described in this section are not prior art to the claims in this application and are not admitted as prior art by inclusion in this section.

Patients in respiratory stress are treated either through manual resuscitators (also referred to as self-inflating bags or bag valve masks) or ventilators (also referred to as respirators). Manual respirators are used to treat acute conditions and to stabilize patients. Ventilators, on the other hand, are typically used for longer term treatment. While manual resuscitators are typically simpler and low-cost devices, ventilator systems are complex and high-cost machines.

### SUMMARY

The present disclosure generally describes a portable, motorized, bag valve mask based respirator that can be used for acute resuscitation and/or longer term patient treatment.

According to some examples, a portable respirator may include an inflatable bag and a respirator body. The inflatable bag may be arranged to provide air to a patient through a face mask or an endotracheal tube in response to compression/retraction actions applied to the inflatable bag. The respirator body may include a first cover portion and a second cover portion arranged to enclose moving components of the portable respirator and define a substantially circular opening for the inflatable bag to be fitted through, a motor, a pair of levers positioned on opposing sides of the substantially circular opening, and a cam mechanically coupled to the motor and arranged to move the pair of levers in response to a rotation action by the motor such that the pair of levers apply the compression/retraction actions to the inflatable bag fitted through the opening.

According to other examples, a portable respirator enclosure may include a first cover portion and a second cover portion, where the first and second cover portions are arranged to enclose moving components of the portable respirator enclosure, the first and second cover portions define a substantially circular opening for an inflatable bag to be fitted through, and the inflatable bag is arranged to provide air to a patient through a face mask or endotracheal tube in response to compression/retraction actions applied to the inflatable bag. The portable respirator enclosure may also include a motor, a pair of levers positioned on opposing sides of the opening, each of the levers including a protrusion facing an equatorial region of the inflatable bag, and a cam mechanically coupled to the motor and arranged to move the pair of levers in response to a rotation action by the motor such that the pair of levers apply the compression/retraction actions through their respective protrusions to the inflatable bag fitted through the substantially circular opening.

According to further examples, a method to operate a portable respirator may include receiving a rotational action from an on-board motor at an oblong shaped cam and in response to a rotation of the cam, moving a pair of levers in a cyclical motion, where the pair of levers are rotatably affixed to inside surfaces of a first and a second cover portion of a respirator enclosure at opposing boundaries of a substantially circular opening defined by the first and second cover portions for an inflatable bag to fit through, and each of the levers comprise a protrusion facing an equatorial region of the inflatable bag. The method may also include reaching a retracted state during a first portion of the cyclical motion, where the inflatable bag is inflated in the retracted state, and reaching a compressed state during a second portion of the cyclical motion, where the protrusions press on an equatorial region of the inflatable bag in the compressed state and force air inside the inflatable bag to be provided to a patient through a face mask or an endotracheal tube.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates a perspective view of an example portable respirator with a mask;

FIG. 2 illustrates two example configurations of an example portable respirator on a patient;

FIG. 3 illustrates a side view of an example portable respirator;

FIG. 4 illustrates an assembly view of the example portable respirator with the cam and levers visible;

FIG. 5 illustrates a mask side view of an example portable respirator with the levers in retracted position;

FIG. 6 illustrates a mask side view of an example portable respirator with the levers in compressed position, arranged in accordance with at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. The aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems and/or devices related to providing a portable, motorized, bag valve mask based respirator that can be used for acute resuscitation and/or longer term patient treatment.

A bag valve mask, also known by the proprietary name Ambu bag® or generically as a manual resuscitator or self-inflating bag, is a hand-held device commonly used to provide positive pressure ventilation to patients who are not breathing or not breathing adequately. The device is used in out-of-hospital settings (such as ambulance crews) and in hospitals as part of standard equipment found on a crash cart, in emergency rooms or other critical care settings. Manual resuscitators are also used for temporary ventilation of patients. The bag valve mask includes a flexible air chamber (the bag), attached to a face mask via a shutter valve. When the face mask is properly applied and the bag is squeezed, the device forces air through into the patient's lungs; when the bag is released, it self-inflates from its other end, drawing in either ambient air or a low pressure oxygen flow supplied by a regulated cylinder, while also allowing the patient's lungs to deflate to the ambient environment (not the bag) past the one way valve.

A ventilator is a machine that provides mechanical ventilation by moving breathable air into and out of the lungs, to deliver breaths to a patient who is physically unable to breathe or breathing insufficiently. Modern ventilators are computerized microprocessor-controlled machines, and are used in intensive care medicine, home care, emergency medicine, and anesthesiology (as a component of an anesthesia machine). In its simplest form, a modern positive pressure ventilator includes a compressible air reservoir or turbine, air and oxygen supplies, a set of valves and tubes, and a disposable or reusable patient circuit. The air reservoir is pneumatically compressed several times a minute to deliver room-air, or in most cases, an air/oxygen mixture to the patient. If a turbine is used, the turbine pushes air through the ventilator, with a flow valve adjusting pressure to meet patient-specific parameters. When over pressure is released, the patient will exhale passively due to the lungs' elasticity, the exhaled air being released usually through a one-way valve within the patient circuit called the patient manifold. Ventilators may also be equipped with monitoring and alarm systems for patient-related parameters (e.g. pressure, volume, and flow) and ventilator function (e.g. air leakage, power failure, mechanical failure), backup batteries, oxygen tanks, and remote control.

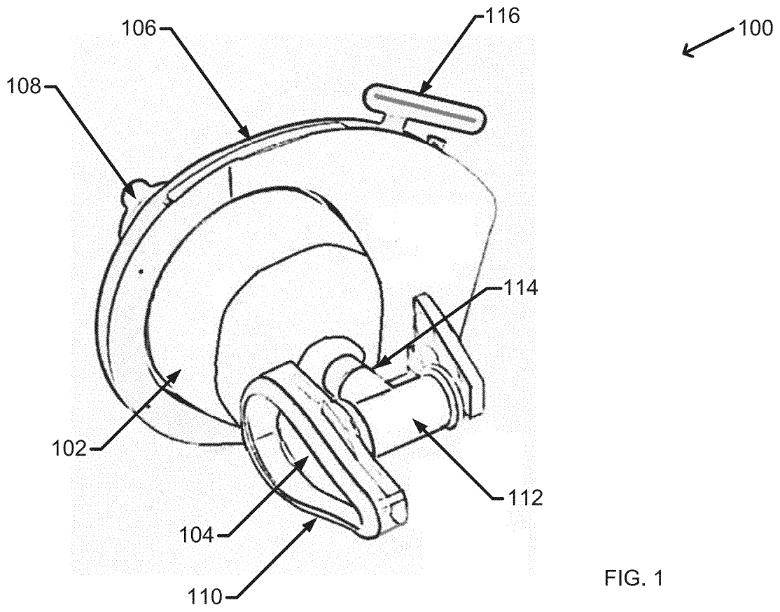

FIG. 1 illustrates a perspective view of an example portable respirator with a mask, arranged in accordance with at least some embodiments described herein.

Diagram 100 shows a portable respirator with mask 110, which provides pressurized air from the bag 102 to the patient's mouth through pipe 112 and air outlet 104. The mask 110 may be rotatable relative to the bag through a swivel 114. The bag 102 is centered in a hole in the enclosure 106, which may include a handgrip 116.

As shown in diagram 100, the inflatable bag 102, which may be made from an elastic material (PVC, polyurethane, etc.) may be inserted into the substantially circular aperture of the portable respirator's enclosure 106. The inflatable bag 102 may be held in place by pressure or may include a pair of ridges around its equatorial region to prevent sliding out in either direction. One side of the inflatable bag (the side indicated by reference numeral 102) may be attached to a mask 110 to provide air to the patient. The mask 110 may be a face mask or a smaller mask covering mouth and nose of the patient. The connection to the face mask (pipe 112) may be removable to allow replacement of the mask and/or bag. For example, different sizes (e.g., pediatric, adult) of masks may be used by simply connecting them to the pipe 112. The other side 108 of the inflatable bag may include an opening to provide air from the environment or connection(s) to provide various concentrations of oxygen from an external oxygen supply (e.g., a tank, a building oxygen distribution system, etc.).

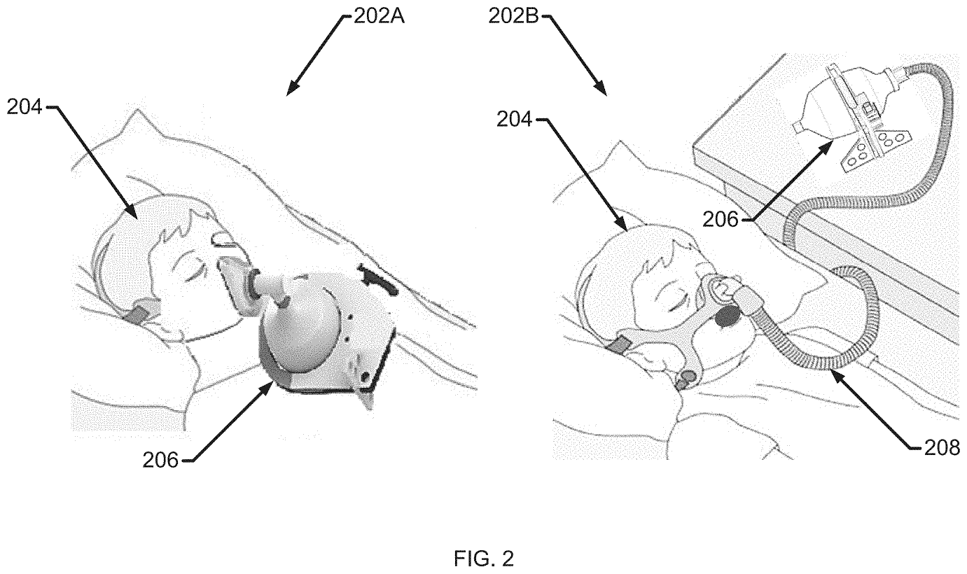

FIG. 2 illustrates two example configurations of an example portable respirator on a patient, arranged in accordance with at least some embodiments described herein.

Diagram 202A in FIG. 2 shows a mask configuration of an example portable respirator 206 on a patient 204. Diagram 202B in FIG. 2 shows an endotracheal tube configuration, where the portable respirator 206 may be coupled to an endotracheal tube via solid tubing or soft tubing and provide respiration to an intubated patient 204. One side of the inflatable bag may be attached to the endotracheal tube to provide air to the patient. The respirator may be affixed to a crash cart or similar fixed location. As with the mask configuration, the other side of the inflatable bag may include an opening to provide air from the environment or connection(s) to provide various concentrations of oxygen from an external oxygen supply (e.g., a tank, a building oxygen distribution system, etc.).

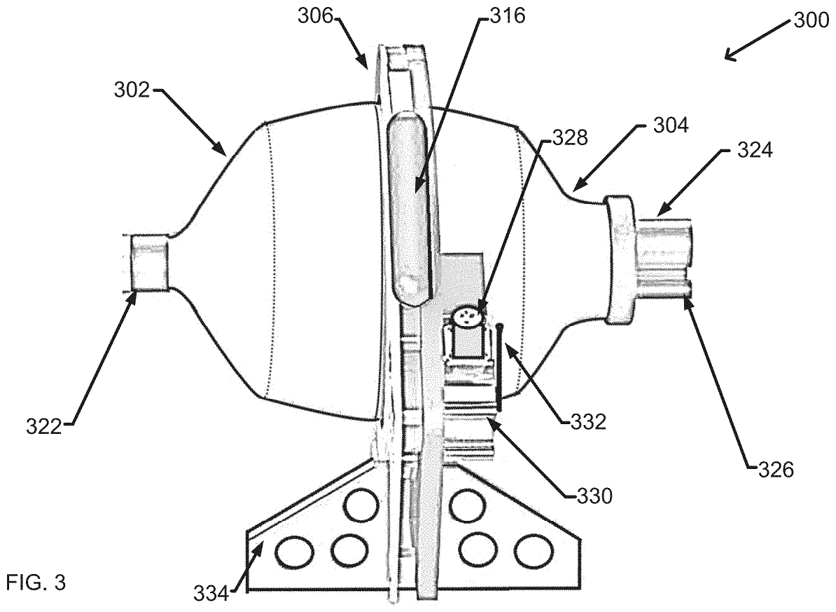

FIG. 3 illustrates a side view of an example portable respirator, arranged in accordance with at least some embodiments described herein.

Diagram 300 shows the portable respirator with removable, keyed stand 334 attached or integrated to the enclosure 306, which straddles the bag. One side 302 of the bag includes the respiratory connection 322 to a mask or endotracheal tube. Another side of the bag includes intake connections 324 and 326. In some examples, a preset or configurable concentration of oxygen may be provided to the inflatable bag. Thus, more than one intake connection may be used to provide oxygen, nitrogen, etc. In other examples, environment air may be provided through one or both of the intake connections 324, 326. In yet other examples, gaseous medication (e.g., asthma inhaler medication) may be added to the provided air. The enclosure 306 may also include (attached or integrated) a motor 330 to inflate and deflate the bag through levers inside the enclosure. The motor 330 may be activated by a manual switch 332. In other embodiments, a connector 328 may be included to provide power to the motor 330 and/or control signals. Thus, the motor 330 may also be remotely activated and controlled (e.g., speed of inflation/deflation). The motor may also be powered by an on-board rechargeable or replaceable battery.

An example portable respirator involves simple and reliable mechanical operation without multiple controls that may require special training. Simple and smooth enclosure may allow preservation of sterility and quick disinfection. With the handgrip 316 and a removable, keyed stand 334, the respirator may be used in-hand (a healthcare professional holding the device over the mouth of a patient) or affixed to a crash cart or other location (connected to the mask or endotracheal tube through tubing) as shown in FIGS. 1 and 2.

In some examples, the motor 330 may be a direct current (DC) motor to move a cam/lever structure. High reliability of the DC motor and connectivity to an external power source may allow easy transition between environments (e.g., from ambulance to hospital, etc.) and reliable power supply (battery pack, vehicle power system, alternative current (AC) converters, etc.). In some examples, the inflatable bag may be replaceable by pushing the bag through the substantially circular aperture of the enclosure 306 to install or to remove. Other types of motors may also be used to actuate the cam/lever system. Connector 328 may be a standardized connector to allow any external power supply such as a battery, a converter, a vehicle or building DC power supply, etc. to be used to power the motor 330. as well as, to provide any external control signals to the motor.

The inflatable bag, which may be made from an elastic material (PVC, polyurethane, etc.) may be inserted into the substantially circular aperture of the portable respirator's enclosure 306 as mentioned above. The inflatable bag may be held in place by pressure or may include a pair of ridges around its equatorial region to prevent sliding out in either direction.

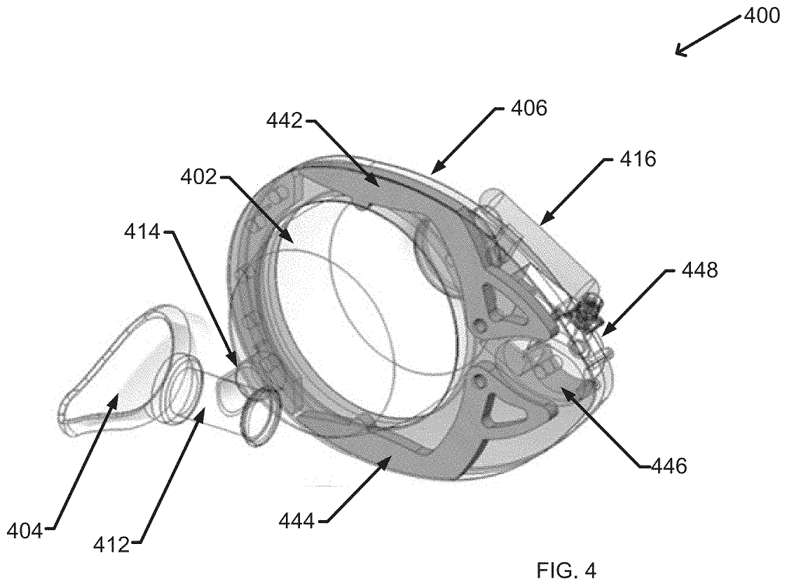

FIG. 4 illustrates an assembly view of the example portable respirator with the cam and levers visible, arranged in accordance with at least some embodiments described herein.

Diagram 400 shows levers 442 and 444 sandwiched between two parts (covers) of the enclosure 406 and controlled by a cam 446 to provide compression and retraction of an inflatable bag 402. Handgrip 416 allows the respirator to be held in the hand of a healthcare professional while the motorized action provides air to a patient through the mask 404. Pipe 412 and swivel 414 may be designed to allow replacement of the mask (e.g., in between uses or with different sizes and shapes of masks) and adjustment of an angle of the mask for ease of operation.

In an operation, the motor 448 may rotate the cam 446 at a predefined speed. The rotation of the cam 446 may, in turn, cause a periodic motion of the levers 442, 444 pushing an equatorial region of the bag 402, thereby causing the bag to be compressed and retracted cyclically. With the levers 442, 444 hidden inside the enclosure 406, catching of hazards (e.g., entangling with tubes) or damage to the levers during operation can be avoided. Due to simplicity of the number and shapes of its components, an example portable respirator may be manufactured from a variety of materials suitable for use in various environments.

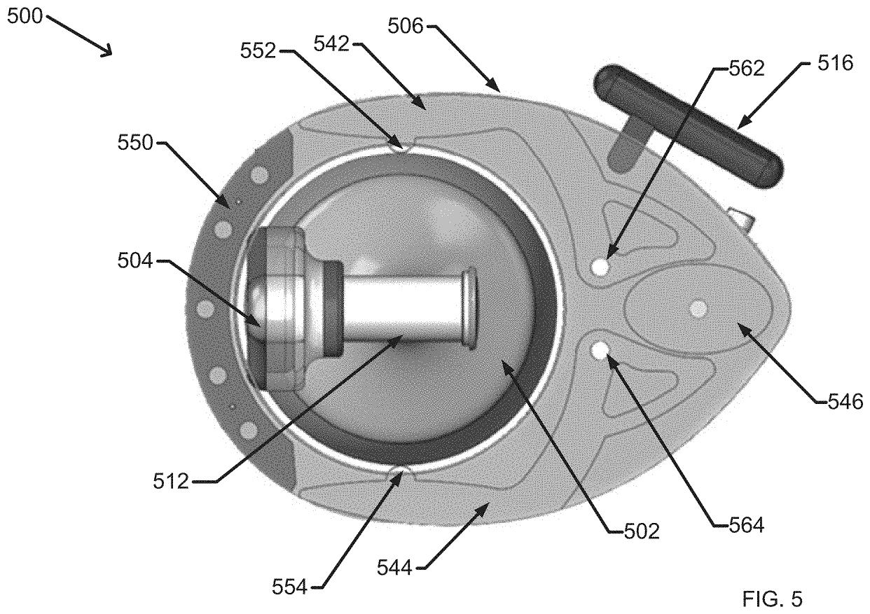

FIG. 5 illustrates a mask side view of an example portable respirator with the levers in retracted position, arranged in accordance with at least some embodiments described herein.

Diagram 500 shows handgrip 516, mask 504, pipe 512, and bag 502, along with inside components of the enclosure 506. The inside components, sandwiched between two covers of the enclosure 506 include cam 546, first lever 542, rotation pin 562 and protrusion 552 of the first lever 542, second lever 544, rotation pin 564 and protrusion 554 of the second lever 544, and pinch guard 550.

The cam 546 may have an oblong shape and be rotated around its rotation pin by a motor (on the opposite side of the enclosure 506). The motion and shape of the cam 546 may cause the first and second levers 542, 544 to move in a cyclical motion around their respective rotation pins and the protrusions 552, 554 to compress and retract an equatorial region of the bag 502. The pinch guard 550 may prevent the bag from expanding toward the front of the enclosure in a compressed state and get pinched between the covers. In the shown retracted state, the protrusions 552, 554 are away from the bag 502 allowing the bag to be fully inflated (with air from the environment or gas such as oxygen from a gas source).

In some examples, the cam/lever system may perform two cycles of retraction/compression per revolution. The average respiration rate for an adult is in a range of 12 to 20 breaths per minute, whereas in infants, the rate may go up to 40 breaths per minute. A potentiometer coupled to the motor may be used to adjust the breath rate (retraction/compression cycles) manually (e.g., through a turn-knob, a slide control, etc.). Alternatively, a speed of the motor may be controlled by electronic signals remotely.

In a normal breath, 500 and 800 milliliters of air may be needed by an adult. The volume of air may be reduced to 400 milliliters or even less if supplemental oxygen is used. Thus, a size of the inflatable bag may vary for adults and children (or even large adults and small adults). In some examples, the portable respirator enclosure (and the cam/lever system) may be made in different sizes to accommodate different sizes of inflatable bags. In other examples, the portable respirator enclosure (and the cam/lever system) may be adjustable for different diameters of inflatable bags. Thus, sizes and shapes of the cam 546, levers 542, 544, and protrusions 552, 554 may be selected based on bag size, type, usage scenarios, etc.

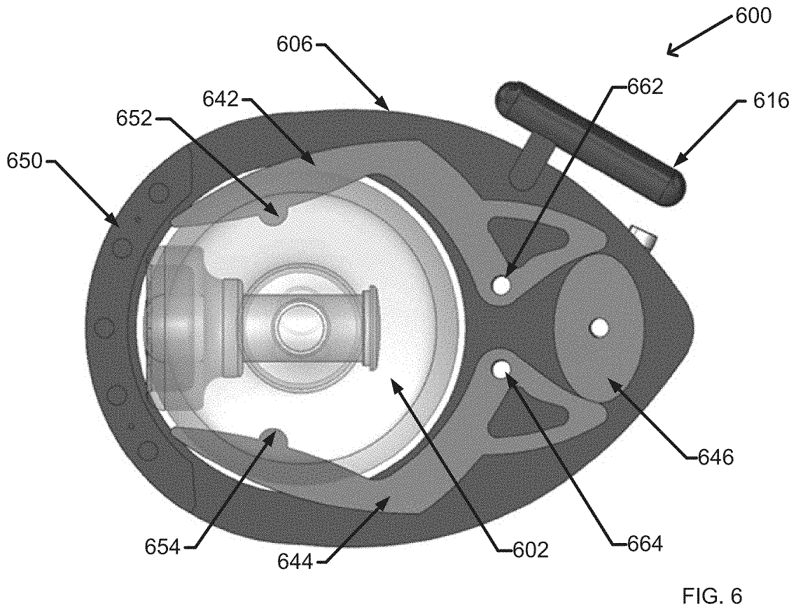

FIG. 6 illustrates a mask side view of an example portable respirator with the levers in compressed position, arranged in accordance with at least some embodiments described herein.

Diagram 600 shows the bag 602 and mask in semi-transparent form to allow visibility of the inside components of the enclosure 606. The inside components include cam 646, first lever 642, rotation pin 662 and protrusion 652 of the first lever 642, second lever 644, rotation pin 664 and protrusion 654 of the second lever 644, and pinch guard 650.

The cam 646 is 90 degrees rotated relative to the retracted state. This causes the first and second levers 642 and 644 to move toward the bag 602, where the protrusions 652 and 654 to compress the bag 602. In the compressed state, the bag's volume is reduced and the air (or oxygen) contained in the bag is pushed into the patient's lungs through his/her mouth.

As mentioned above, the cam and the levers are sandwiched between two covers of the enclosure. The sandwiching, their shapes, and mechanical coupling (one example shown in the figures) may prevent the cam and the levers from separating or otherwise becoming loose or disassembled in either retracted or compressed states. The levers and protrusions on the levers may be shaped such that in a compressed state, one hemisphere of the inflatable bag is collapsed simulating manual compression of the bag. The cam may have an oblong or other shape. In some examples, two orthogonal axes of the cam may correspond to the compressed and retracted states of the levers, respectively.

The cam, levers, and/or the covers of the enclosure may be made from various synthetic materials such as PVC, polypropylene, ceramic, or metals such as aluminum, stainless steel, and other materials. In one example implementation, the enclosure of the portable respirator may have a depth (distance between the surfaces of the two covers) of about 1.25 inches and the overall respirator may be about 11 inches wide and about 17 inches long. The keyed stand may be removable to allow even easier use in the manual mode and may include a number of predrilled holes to allow affixing of the respirator to fixed locations.

According to some examples, a portable respirator may include an inflatable bag arranged to provide air to a patient through a face mask or an endotracheal tube in response to compression/retraction actions applied to the inflatable bag and a respirator body. The respirator body may include a first cover portion and a second cover portion, the first and second cover portions arranged to enclose moving components of the portable respirator and define a substantially circular opening for the inflatable bag to be fitted through; a motor; a pair of levers positioned on opposing sides of the substantially circular opening; and a cam mechanically coupled to the motor and arranged to move the pair of levers in response to a rotation action by the motor such that the pair of levers apply the compression/retraction actions to the inflatable bag fitted through the opening.

According to other examples, the portable respirator may also include a handgrip affixed to the respirator body; a keyed stand element arranged to allow attachment of the respirator body to a fixed location; a face mask affixed to the inflatable bag through hard tubing; and/or an endotracheal tube affixed to the inflatable bag through soft tubing. The inflatable bag may include a first connector to allow attachment of the face mask or the endotracheal tube to one end of the inflatable bag and a second connector to allow air intake at another end of the inflatable bag. The portable respirator may further include a third connector to allow concentrated oxygen intake at the other end of the inflatable bag. The portable respirator may also include a portable power supply electrically coupled to the motor through a connector and/or a speed adjustment circuit electrically coupled to the motor and arranged to adjust a frequency of the compression/retraction actions.

According to further examples, a portable respirator enclosure may include a first cover portion and a second cover portion, where the first and second cover portions are arranged to enclose moving components of the portable respirator enclosure, the first and second cover portions define a substantially circular opening for an inflatable bag to be fitted through, and the inflatable bag is arranged to provide air to a patient through a face mask or endotracheal tube in response to compression/retraction actions applied to the inflatable bag. The enclosure may also include a motor; a pair of levers positioned on opposing sides of the opening, each of the levers comprising a protrusion facing an equatorial region of the inflatable bag; and a cam mechanically coupled to the motor and arranged to move the pair of levers in response to a rotation action by the motor such that the pair of levers apply the compression/retraction actions through their respective protrusions to the inflatable bag fitted through the substantially circular opening.

According to some examples, the portable respirator enclosure may further include a keyed stand element arranged to allow attachment of the respirator body to a fixed location and/or a handle grip. A shape of the protrusion may be selected to compress one hemisphere of the inflatable bag in a compressed state. The cam may have an oblong shape with each axis of the oblong shape corresponding to one of a compressed state and a retracted state. The motor may be a direct current (DC) motor powered by an on-board battery or a remote power supply. The portable respirator enclosure may also include a speed adjustment circuit electrically coupled to the motor and arranged to adjust a frequency of the compression/retraction actions. The speed adjustment circuit may be controlled by a control element affixed to the portable respirator enclosure or a remote device. A size and/or a shape of the substantially circular opening may be adjustable to allow different sizes of inflatable bags to be fitted.

According to other example, a method to operate a portable respirator may include receiving a rotational action from an on-board motor at an oblong shaped cam; in response to a rotation of the cam, moving a pair of levers in a cyclical motion, where the pair of levers are rotatably affixed to inside surfaces of a first and a second cover portion of a respirator enclosure at opposing boundaries of a substantially circular opening defined by the first and second cover portions for an inflatable bag to fit through, and each of the levers comprise a protrusion facing an equatorial region of the inflatable bag. The method may further include reaching a retracted state during a first portion of the cyclical motion, where the inflatable bag is inflated in the retracted state; and reaching a compressed state during a second portion of the cyclical motion, where the protrusions press on an equatorial region of the inflatable bag in the compressed state and force air inside the inflatable bag to be provided to a patient through a face mask or an endotracheal tube. The method may also include adjusting a frequency of compression/retraction actions by adjusting a speed of the motor in response to a manual input through a control element on the portable respirator enclosure or a control signal from a remote device. The retracted state and the compressed state may correspond to two orthogonal axes of the cam.

The present disclosure is not to be limited in terms of the particular embodiments described in this application, which are intended as illustrations of various aspects. Many modifications and variations can be made without departing from its spirit and scope. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled. The terminology used herein is for the purpose of describing particular embodiments only and is not intended to be limiting.

The herein described subject matter sometimes illustrates different components contained within, or connected with, different other components. Such depicted architectures are merely examples, and in fact, many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments are possible. The various aspects and embodiments disclosed herein are for purposes of illustration and are not intended to be limiting, with the true scope and spirit being indicated by the following claims.

What is claimed is:

1. A portable respirator comprising:
an inflatable bag arranged to provide air to a patient through a face mask or an endotracheal tube in response to compression/retraction actions applied to the inflatable bag; and
a respirator body comprising:
a first cover portion and a second cover portion, the first and second cover portions arranged to enclose moving components of the portable respirator and define a substantially circular opening for the inflatable bag to be fitted through;
a motor;
a pair of levers positioned on opposing sides of the substantially circular opening; and
a cam mechanically coupled to the motor and arranged to move the pair of levers in response to a rotation action by the motor such that the pair of levers apply the compression/retraction actions to the inflatable bag fitted through the opening.

2. The portable respirator of claim 1, further comprising:
a handgrip affixed to the respirator body.

3. The portable respirator of claim 1, further comprising:
a keyed stand element arranged to allow attachment of the respirator body to a fixed location.

4. The portable respirator of claim 1, further comprising:
a face mask affixed to the inflatable bag through hard tubing.

5. The portable respirator of claim 1, further comprising:
an endotracheal tube affixed to the inflatable bag through soft tubing.

6. The portable respirator of claim 1, wherein the inflatable bag comprises:
a first connector to allow attachment of the face mask or the endotracheal tube to one end of the inflatable bag; and
a second connector to allow air intake at another end of the inflatable bag.

7. The portable respirator of claim 6, further comprising:
a third connector to allow concentrated oxygen intake at the other end of the inflatable bag.

8. The portable respirator of claim 1, further comprising:
a portable power supply electrically coupled to the motor through a connector.

9. The portable respirator of claim 1, further comprising:
a speed adjustment circuit electrically coupled to the motor and arranged to adjust a frequency of the compression/retraction actions.

10. A portable respirator enclosure comprising:
a first cover portion and a second cover portion, wherein
the first and second cover portions are arranged to enclose moving components of the portable respirator enclosure,
the first and second cover portions define a substantially circular opening for an inflatable bag to be fitted through, and
the inflatable bag is arranged to provide air to a patient through a face mask or endotracheal tube in response to compression/retraction actions applied to the inflatable bag;
a motor;
a pair of levers positioned on opposing sides of the opening, each of the levers comprising a protrusion facing an equatorial region of the inflatable bag; and
a cam mechanically coupled to the motor and arranged to move the pair of levers in response to a rotation action by the motor such that the pair of levers apply the compression/retraction actions through their respective protrusions to the inflatable bag fitted through the substantially circular opening.

11. The portable respirator enclosure of claim 10, further comprising:
a keyed stand element arranged to allow attachment of the respirator body to a fixed location; and/or
a handle grip.

12. The portable respirator enclosure of claim 10, wherein a shape of the protrusion is selected to compress one hemisphere of the inflatable bag in a compressed state.

13. The portable respirator enclosure of claim 10, wherein the cam has an oblong shape with each axis of the oblong shape corresponding to one of a compressed state and a retracted state.

14. The portable respirator enclosure of claim 10, wherein the motor is a direct current (DC) motor powered by an on-board battery or a remote power supply.

15. The portable respirator enclosure of claim 10, further comprising:
a speed adjustment circuit electrically coupled to the motor and arranged to adjust a frequency of the compression/retraction actions.

16. The portable respirator enclosure of claim 15, wherein the speed adjustment circuit is controlled by a control element affixed to the portable respirator enclosure or a remote device.

17. The portable respirator enclosure of claim 10, wherein a size and/or a shape of the substantially circular opening is adjustable to allow different sizes of inflatable bags to be fitted.

18. A method to operate a portable respirator, the method comprising:
receiving a rotational action from an on-board motor at an oblong shaped cam;
in response to a rotation of the cam, moving a pair of levers in a cyclical motion, wherein
the pair of levers are rotatably affixed to inside surfaces of a first and a second cover portion of a respirator enclosure at opposing boundaries of a substantially circular opening defined by the first and second cover portions for an inflatable bag to fit through, and
each of the levers comprise a protrusion facing an equatorial region of the inflatable bag;
reaching a retracted state during a first portion of the cyclical motion, wherein the inflatable bag is inflated in the retracted state; and
reaching a compressed state during a second portion of the cyclical motion, wherein the protrusions press on an equatorial region of the inflatable bag in the compressed state and force air inside the inflatable bag to be provided to a patient through a face mask or an endotracheal tube.

19. The method of claim 18, further comprising:
adjusting a frequency of compression/retraction actions by adjusting a speed of the motor in response to a manual input through a control element on the portable respirator enclosure or a control signal from a remote device.

20. The method of claim 18, wherein the retracted state and the compressed state correspond to two orthogonal axes of the cam.
